(19) United States
(12) Patent Application Publication
(10) Pub. No.: US 2010/0160224 A1
(43) Pub. Date: Jun. 24, 2010

(54) SHELF-STABLE CONSUMABLE COMPOSITIONS CONTAINING PROBIOTIC-MIMICKING ELEMENTS AND METHODS OF PREPARING AND USING THE SAME

(76) Inventors: David Thomas, Frisco, CA (US); James Steele, Madison, WI (US)

Correspondence Address: COX SMITH MATTHEWS INCORPORATED, 112 EAST PECAN STREET, SUITE 1800, SAN ANTONIO, TX, 78205-1521, US

(21) Appl. No.: 12/579,176

(22) Filed: Oct. 14, 2009

Related U.S. Application Data
(60) Provisional application No. 61/105,208, filed on Oct. 14, 2008.

(51) Int. Cl. A61K 38/14; A61K 38/00; A61K 38/02; A61K 31/715; A61K 31/66; C08K 5/00; A61K 31/20; C07K 2/00; C07H 21/00; C07C 53/00; C07F 9/02; C07F 9/06; A61P 1/14

(52) U.S. Cl. 514/12; 514/2; 514/44 R; 514/53; 514/54; 514/75; 514/102; 514/558; 530/350; 536/23.7; 554/1; 554/78; 562/8

(57) ABSTRACT

A shelf-stable consumable composition may comprise one or more Probiotic-Mimicking Elements (PMEs) wherein the median particle size of the one or more PMEs is less than the median particle size of a probiotic from which the one or more PMEs were derived. In some embodiments, the median particle size of the one or more PMEs may be less than about 0.2 microns. In some embodiments, the PMEs may comprise at least one of peptides, fatty acids, bacteriocins, cell surface proteins, phospholipids, teichoic acids, and nucleic acids, and may be combined with one or more prebiotics, such as oligosaccharides, inulin, short-chain fructo-oligosaccharides, xylooligosaccharides, galactooligosaccharides, soyoligosaccharides, and lactulose/lactitol. Methods for preparing and using a shelf-stable consumable composition for promoting digestive health in a subject are also described.

### CROSS-REFERENCE TO RELATED APPLICATIONS

This application claims priority to U.S. Provisional Patent Application No. 61/105,208 filed on Oct. 14, 2008.

### FIELD

This application relates generally to shelf-stable consumable compositions containing Probiotic-Mimicking Elements (PMEs) and methods for using the same.

### BACKGROUND

A probiotic is a potentially beneficial organism which may populate the digestive system of an individual. The best-known probiotic species is Lactobacillus acidophilus, which is found in yogurt, acidophilus milk, and dietary supplements. A number of different organisms have been identified as probiotic, though the most commonly used probiotic species are bacterial and may include bacteria that produce lactic acid via the metabolism of carbohydrates, such as strains of Lactobacillus and Bifidobacterium. These bacteria are readily cultured, non-toxic and may be used in many commercial products.

Examples of probiotic bacterial species may include, but are not limited to, Bifidobacterium animalis subsp. lactis (Bb12™, Chr. Hansen; Howaru Bifido™, Danisco), Bifidobacterium breve (Bifiene™, Yakult), Bifidobacterium infantis (Align™, Procter & Gamble), Bifidobacterium longum (Morinaga Milk Industry), Escherichia coli (ProBactrix™, BioBalance; Mutaflor™, Ardeypharm), Lactobacillus acidophilus (NCFM, Danisco, Chr. Hansen), Lactobacillus casei (Actimel™ or DanActive™, Danone; Cultura™, Aria Foods; Yakult™, Yakult), Lactobacillus paracasei (Nestlé), Lactobacillus johnsonii (Nestlé), Lactococcus lactis (Norrmejerier), Lactobacillus plantarum (GoodBelly™, ProViva™, or TuZen™, NextFoods), Lactobacillus reuteri (BioGaia Biologics), Lactobacillus rhamnosus (Vifit™, Valio and Verum™, Norrmejerier), and Saccharomyces cerevisiae (DiarSafe™, Wren Laboratories).

Probiotic bacterial cultures assist the body's naturally-occurring ecology of microbes by preventing the growth of harmful or otherwise undesirable microbes within the digestive system. Probiotics that survive processing and ingestion may attach to the gastrointestinal epithelium of an individual and thereby compete with less common and potentially detrimental bacteria that may become prominent in a diseased or unhealthy state. In competing for available sites within the gut, probiotics help maintain a healthy balance of bacteria within the digestive system. Probiotic bacteria have developed many different mechanisms to compete with co-habitating species. These mechanisms include the induction of an immune mediated response in the host organism, modification of intestinal pH and saturation of the environment with selective nutrients.

Studies suggest that probiotics may be effective in treating numerous disorders and in promoting general health. For example, probiotics have been found to lessen the effects of lactose intolerance by converting lactose into lactic acid. See Sanders, M. E; Considerations for use of probiotic bacteria to modulate human health; J. Nutr. 130 (2S Suppl.) February 2003; 84S-390S. Some strains of Lactobacilli have also demonstrated anti-mutagenic effects arising from their ability to bind with certain carcinogens formed in cooked meat. See Wollawski, I; Protective role of probiotics and prebiotics in colon cancer, Am. J. Clin. Nutr. 73 (2 Suppl.) February 2001; 451S-455S. Animal studies have even shown that some probiotics can protect against colon cancer, and human trials suggest further anti-carcinogenic effects arising from a decrease in the activity of enzymes (e.g., β-glucuronidase), which can generate carcinogens from procarcinogenic compounds in the digestive system. See Brady, L. J.; The role of probiotic cultures in the prevention of colon cancer, J. Nutr. 130 (2SSuppl.) February 2000; 410S-414S.

Other medical studies suggest that probiotics may have beneficial effects on the blood. For example, animal studies have demonstrated the lowering of blood cholesterol through administration of probiotics, which break down bile in the digestive system before it can be reabsorbed in the blood as cholesterol. See Sanders, M. E.; Considerations for use of probiotic bacteria to modulate human health; J. Nutr. 130 (2S Suppl.) February 2003; 84S-390S. Clinical trials have also shown that probiotics may reduce blood pressure through the production of certain ACE inhibitor-like peptides. See id.

Probiotics are also thought to have several beneficial effects on the immune function. For example, probiotics are known to modulate the host immune system via interactions with the gut associated lymphoid tissue (GALT). The outcomes of these interactions are numerous and include anti-inflammatory responses, regulation of allergic reactions, and enhanced resistance to infection by intestinal and respiratory pathogens. For example, probiotics have been shown to protect against numerous pathogens while increasing the number if IgA-producing plasma cells, T lymphocytes, and Natural Killer cells. See Reid G.; Potential uses of probiotics in clinical practice; Clin. Microbial Rev. 16(4) October 2003; 658-72. Clinical trials suggest that probiotics may even decrease the incidence of respiratory tract infections. See Hatakka K.; Effect of long term consumption of probiotic milk on infections in children attending day care centres: double blind, randomized trial; BMJ 322 (7298) June 2001; 1327. Similarly, probiotics have been shown to be effective in treating rotavirus infections in children and various types of diarrhea, such as acute diarrhea, traveler's diarrhea, and antibiotic-associated diarrhea. See Ouwehand A. C.; Probiotics: an overview of beneficial effects; Antonie Van Leeuwenhoek 82 (1-4) August 2002; 279-89; see also Cremonini F.; Meta-analysis: the effect of probiotic administration on antibiotic-associated diarrhea; Aliment. Pharmacol. Ther. 16 (8) August 2002; 1461-7.

Researchers have shown that heat killed bacteria may initiate an immune response in human subjects. For example, flow cytometry has been used to analyze an immune response through the monitoring of the differentiation profile of peripheral blood mononuclear cells isolated from patients that were provided a heat killed sample of a Lactobacilus plantarum strain (L-137). See Hirose, Y.; Daily Intake of Heat-Killed Lactobacillus plantarum L-137 Augments Acquired Immunity in Healthy Adults: J. Nutr. (136) 2006: 3069-3073. The induction of innate immunity may be linked to a suppression of antibodies associated with food allergies. See Murosaki, S.; Heat-Killed Lactobaciullus plantarum L-137suppresses naturally fed antigen-specific IgE production by stimulation of IL-12 production in mice: J Allergy Clin Immunol. (102) 1998: 57-64. In other studies heat-killed Lactobacillus platarum (L-137) was provided to mice by intraperitoneal injection, and anti-tumor effects were found in at least some circumstances. See Murosaki, S.; Antitumor effect of heat-killed Lactobacillus plantarum L-137 through restoration of impaired interleukin-12 production in tumor-bearing bearing mice: Cancer Immunol. Immunother. (49) 2000: 157-164. Numerous other digestive disorders have been shown to respond to the consumption of probiotics. For example, probiotics have been found to moderate inflammation due to regulation of the cytokine function, which may prevent recurrence of inflammatory bowel disease, improve milk allergies, and decrease the risk of atopic eczema in children. See Reid G.; Potential uses of probiotics in clinical practice; Clin. Microbial Rev. 16(4) October 2003; 658-72; see also Kalliomaki M.; Probiotics and prevention of atopic disease: 4-year follow-up of a randomized placebo-controlled trial; Lancet 361 (9372) May 2003; 1869-71. Adults suffering from Helicobacter pylori infections, which cause peptic ulcers, have also been shown to benefit from ingestion of probiotics. See Hamilton-Miller J. M.; The role of probiotics in the treatment and prevention of Helicobacter pylori infection; Int. J. Antimicrob. Agents 22 (4) October 2003; 360-6.

In addition to treating various disorders, probiotics have been found to promote beneficial processes within the body, such as mineral absorption. For example, studies suggest that probiotics may help correct malabsorption of trace minerals. See Famularo G.; Probiotic lactobacilli: an innovative tool to correct the malabsorption syndrome of vegetarians?; Med. Hypotheses 65 (6) 2005; 1132-5.

While products containing probiotics have many beneficial effects, probiotics as living cultures do impose certain restrictions in regards to processing and packaging due to shortened shelf-life. For example, the most common commercial probiotic-containing products include yogurt and other dairy products which must remain refrigerated and have a limited shelf-life. If such products are subjected to extremes of heat and/or excessive storage time, the population of viable cultures decreases and product utility and effectiveness may be lost or severely compromised

Additionally, there exist prebiotic substances which may promote the growth of probiotics already present in the gut. Examples of prebiotic substances are oligosaccharides such as fructo-oligosaccharide (FOS) and galacto-oligosaccharide (GOS), inulin (Imperial Holly Corp., Sugar Land, Tex.), NutraFlora, which is a short-chain fructo-oligosaccharide (Golden Technologies, Westminister, Colo.), xylooligosaccharides, galactooligosaccharides, soyoligosaccharides, and lactulose/lactitol.

Probiotics may secrete certain antibacterial peptides which have been shown to regulate microbial ecology within the body. See Silva, M.; Antimicrobial substance from a human Lactobacillus strain; Antimicrob. Agents Chemother 31(8) August, 1987; 1231-33. Additionally, when probiotics die, peptides may be released through the degradation of their cellular walls. D-peptiodoglycan is a peptide-containing component of bacterial cell walls which has been shown to initiate an immune response. See Vanderpool, C.; Inflammatory Bowel Diseases; Inflamm. Bowel Dis. 2008.

Bacteriocins are a diverse class of protein-based materials which are used by probiotic species to compete with similar bacteria, and bacteriocins produced by species which commonly inhabit the gut play an important protective role against harmful bacteria. A range of different bacteriocins has been studied for various therapeutic means, both as general antimicrobial agents (Teather, U.S. Pat. No. 6,255,080; Dobrogoz, U.S. Pat. No. 5,439,678) and in treatment of cancers such as leukemia. See Musclow, C. E.; Acute lymphoblastic leukemia of childhood monitored by bacteriocins and flowcytometry; Eur J Cancer Clin. Oncol.; 23 (4) April 1987; 411-18.

Bacteriocins have been classified by various means including their mechanism of toxicity, structure, and general stability. For example, Class I bacteriocins are small peptide inhibitors such as nisin. Class II bacteriocins are a group of small proteins that show good heat stability, and are thought to function by deactivating mannose transport in targeted cells. See Dzung, B. D., Common mechanisms of target cell recognition and immunity for class II bacteriocins; Proc Natl Acad. Sci.; v. 104(7) Feb. 13, 2007. However, Class II bacteriocins may work by a variety of other mechanisms, including membrane pore formation, DNA cleavage, and disruption of murein production. An example of a bacteriocin derived from a class of lactobacillus is reuterin, which has been shown to demonstrate antibiotic properties. Class III bacteriocins are large, heat-labile protein bacteriocins. A large number of bacteriocins have been identified, and these compounds may be described through reference to several on-line genomic databases. See de Jong, A.; BAGEL: a web-based bacteriocins genome mining tool; Nucleic Acids Research (34) 2006; W273-W279; see also Hammami, R.; BACTIBASE: a new web-accessible database for bacteriocins characterization; BMC Microbiology (7) 2007; 89.

Fructose and galactose-based oligosaccharides (FOS and GOS) are carbohydrates commonly paired with probiotics to encourage probiotic growth and maintenance. For example, FOS is selectively metabolized by the probiotic strains of bifidobacteria, and FOS is often used as a substrate to facilitate growth of the same. FOS is not metabolized by the body, and any excess of this non-digestible sugar is passed through the digestive system. In view of the relatively short residence time of probiotics within the gut, and the need of consumers to ingest probiotics on a regular basis in order to maintain probiotic cultures, consumption of FOS may help prolong the life of such cultures within the gut and thereby reduce the frequency at which live cultures must be consumed.

### SUMMARY

A shelf-stable consumable composition may comprise one or more Probiotic-Mimicking Elements (PMEs) wherein the median particle size of the PMEs is less than the median particle size of the probiotic(s) from which the PMEs were derived. In some embodiments, the median particle size of the PMEs may be less than about 0.2 microns. In some embodiments, the PMEs may comprise at least one of peptides, fatty acids, bacteriocins, cell surface proteins, phospholipids, teichoic acids, and nucleic acids, and may be combined with one or more probiotics, such as oligosaccharides, inulin, short-chain fructo-oligosaccharides, xylooligosaccharides, galactooligosaccharides, soyoligosaccharides, and lactulose/lactitol. Methods for preparing and using a shelf-stable consumable composition for promoting digestive health in a subject are also described.

### DETAILED DESCRIPTION

As used herein, the following terms should be understood to have the indicated meanings:

When an item is introduced by “a” or “an,” it should be understood to mean one or more of that item.

The terms “first,” “second,” and the like as used herein do not denote any order, quantity, or importance, but rather are used to distinguish one element from another.

“Comprises” means includes but is not limited to.

“Comprising” means including but not limited to.

“Having” means including but not limited to.

“Consumable composition” means any substance that may be ingested by a person or animal. Examples of consumable compositions may include beverages and foods.

“Beverage” means any drinkable liquid or semi-liquid, including for example and without limitation, flavored water, soft drinks, fruit drinks, coffee-based drinks, tea-based drinks, juice-based drinks, milk-based drinks, gel drinks, carbonated and non-carbonated drinks, sauces, alcoholic and non-alcoholic drinks.

“Food-grade acid” means any acid that is acceptable for use in edible compositions.

“Added water” means water added to a beverage as a component, and does not mean water incidentally added to a beverage through other components. The added water may be specifically purified prior to use using processes well-known in the art such as filtration, deionization, distillation, or reverse osmosis.

“Dry composition” means the composition of a beverage without taking into account any added water.

“Liquid composition” means the composition of a beverage including any added water.

“Flavors” mean flavoring agents such as natural flavors, artificial flavors, spices, seasonings, and the like. Exemplary flavoring agents may include synthetic flavor oils and flavoring aromatics and/or oils, oleoresins, essences, distillates, and extracts derived from plants, leaves, flowers, fruits, and so forth, and a combination comprising any of the foregoing.

“Flavor potentiator” means a material that can intensify, supplement, modify or enhance the taste and/or aroma perception of a composition without introducing a characteristic taste and/or aroma perception of its own. Flavor potentiators may supplement, modify, or enhance the perception of flavor, sweetness, tartness, umami, kokumi, saltiness, bitterness, and a combination comprising any of the foregoing, for example.

“Nutrient” means any material capable of being metabolized or otherwise used by the body.

“Probiotic” means a potentially beneficial organism which may populate the digestive system of an individual or animal.

“Inactivate” means to kill an organism.

“Inactivated probiotic” means a probiotic that is no longer living.

“Probiotic metabolite” means a substance secreted or excreted by a probiotic.

“Cellular component” means a constituent part of an inactivated probiotic.

“Prebiotic” means a substance which promotes the growth of a probiotic.

“Probiotic-mimicking element (PME)” means a probiotic metabolite or a cellular component of an inactivated probiotic.

“Bioactive” means having a capacity to interact with a living subject to improve the health of that subject. For example, and without limitation, a bioactive PME may improve a subject's digestion, lessen the effects of lactose intolerance, demonstrate anti-mutagenic effects, lower blood cholesterol, improve the subject's immunological response, or a combination of any of the foregoing.

“Culture” means a group of bacteria.

“Subject” means a human or an animal.

The endpoints of all ranges directed to the same component or property are inclusive and independently combinable.

The present application is directed to a shelf-stable consumable composition containing one or more Probiotic-Mimicking Elements (PMEs). The application is also directed to a method of preparing and using a shelf-stable consumable composition.

PMEs are not living organisms and may therefore be able to withstand the processing (e.g., acidic environment, thermal treatment) and storage conditions required of shelf-stable consumable compositions. Fortification of consumable compositions with one or more PMEs thus provides such compositions with many of the benefits associated with probiotics, while avoiding the limited process sensitivity and shelf-stability of probiotics. Shelf-stable PMEs which do not require refrigeration and can be packaged in a wide range of shelf-stable consumer products may therefore

[symbol]

PMEs may exhibit varying degrees of bioactivity, and some PMEs may not be bioactive. In general, a shelf-stable consumable composition may comprise one or more PMEs wherein the median particle size of the one or more PMEs is less than the median particle size of the one or more probiotics from which the one or more PMEs were derived. It is believed that the most bioactive PMEs have a median particle size of less than about 0.2 microns. The median particle size of PMEs may also affect the transparency of consumable compositions to which the PMEs may be added. As the median particle size of the PMEs increases, the transparency of consumable compositions comprising those PMEs may generally decrease. It is anticipated that PMEs having a median particle size of less than about 0.2 microns may be added to foods, beverages, and various other compositions without resulting in the formation of an observable cloudy haze within the composition. Such compositions could therefore be fortified with PMEs while remaining substantially transparent.

In one embodiment, a consumable composition may be formed which contains one or more PMEs having a median particle size of less than about 0.2 microns. In another embodiment, a consumable composition may be formed which contains one or more PMEs having a median particle size of less than about 0.2 microns, and one or more prebiotics. Suitable PMEs may include but are not limited to peptides, fatty acids, bacteriocins, cell surface proteins, phospholipids, teichoic acids, and nucleic acids. Suitable prebiotics may include but are not limited to oligosaccharides, inulin, short-chain fructo-oligosaccharides, xylooligosaccharides, galactooligosaccharides, soyoligosaccharides, and lactulose/lactitol.

In yet another embodiment, a consumable composition may be formed which contains one or more PMEs in concentrations that are consistent with probiotic metabolism. In another embodiment, a consumable composition may be formed which contains one or more PMEs in concentrations that are lesser than or greater than concentrations consistent with probiotic metabolism. A person having ordinary skill in the art will understand that the concentrations of PMEs that are consistent with probiotic metabolism may depend upon the type of probiotic.

A method of producing a shelf-stable consumable composition comprising one or more PMEs is also disclosed herein. In a first step, bacteria may be grown using various culturing procedures. Any number of bacteria may be cultured, and such procedures may involve maintenance of bacteria in a growth medium, and may involve a growth chamber with a controlled temperature, pH, or other suitable variables. Any number of cultures using any number of growth media may be used. Bacterial cultures that may be grown include, but are not limited to, Bifidobacterium animalis subsp. lactis (Bb12™, Chr. Hansen; Howaru Bifido™, Danisco), Bifidobacterium breve (Bifiene™, Yakult), Bifidobacterium infantis (Align™, Procter & Gamble), Bifidobacterium longum (Morinaga Milk Industry), Escherichia coil (ProBactrix™, BioBalance; Mutaflor™, Ardeypharm), Lactobacillus acidophilus (NCFM, Danisco, Chr. Hansen), Lactobacillus easel (Actimel™ or DanActive™ Danone; Cultura™, Aria Foods; Yakult™, Yakult), Lactobacillus paracasei (Nestlé), Lactobacillus johnsonii (Nestlé), Lactococcus lactis (Norrmejerier), Lactobacillus plantarum (GoodBelly™, ProViva™, or TuZen™, NextFoods), Lactobacillus reuteri (BioGaia Biologics), Lactobacillus rhamnosus (Vifit™, Valio and Verum™, Norrmejerier), and Saccharomyces cerevisiae (DiarSafe™, Wren Laboratories).

In a next step, bacterial cultures may be inactivated using any number of means. For example, bacteria may be inactivated by application of heat, pressure, electromagnetic energy, physical maceration, sonic energy, chemicals, or any combination thereof. In one embodiment, application of heat may involve subjecting bacteria to a temperature of about 90° C. for a time period of about 20 minutes. In another embodiment, electromagnetic energy useful for killing bacteria may involve ionizing or nonionizing radiation. In yet another embodiment, sonic energy may be used to disrupt bacterial cell membranes and may involve the propagation of sound energy through numerous mediums, such as, for example, a liquid or gaseous medium. In another embodiment, chemical disruption of bacteria may involve the application of surfactants, detergents, or any other chemicals capable of breaking down the cellular membranes of the bacteria.

In a next step, inactivated probiotics may be subjected to further processing in order to create one or more PMEs having a particular median particle size. Such processing may include, but is not limited to, particle separation using centrifugation, ultrafiltration, selectively permeable membranes, solvent extraction, preparative chromatography, or any combination thereof. In some embodiments, the one or more PMEs may have a median particle size less than the median particle size of the one or more probiotics from which the one or more PMEs were derived. In one embodiment, inactivated probiotics may be filtered to create PMEs having a median particle size of less than about 0.2 microns.

A person having ordinary skill in the art will understand that PMEs may remain in solution after processing or may be dried through various means, including for example, spray drying, freeze drying, evaporative drying, or any combination thereof, PMEs may further be added to foods, beverages, and various other compositions.

One embodiment of a method by which an individual may use a consumable composition to promote digestive health involves orally administering to the individual an effective amount of a consumable composition as described herein, for example, a consumable composition containing one or more PMEs wherein the median particle size of the one or more PMEs is less than the median particle size of the one or more probiotics from which the one or more PMEs were derived. In some embodiments, such PMEs may have a median particle size of less than about 0.2 microns. Another embodiment of a method by which an individual may use a consumable composition to promote digestive health involves orally administering to the individual an effective amount of a consumable composition containing one or more PMEs such as peptides, fatty acids, bacteriocins, cell surface proteins, phospholipids, teichoic acids, and nucleic acids. In yet another embodiment, one or more PMEs may be combined with one or more prebiotics, such as oligosaccharides, inulin, short-chain fructo-oligosaccharides, xylooligosaccharides, galactooligosaccharides, soyoligosaccharides, and lactulose/lactitol. In yet another embodiment, one or more PMEs and one or more prebiotics may be administered separately to the individual.

A consumable composition as described herein may have any desired amount of added water and may be consumed by an individual via oral administration to promote digestive health.

A person having ordinary skill in the art will understand that embodiments of a consumable composition may contain one or more flavors. Exemplary flavor oils may include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil; useful flavoring agents may include artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, prune, raisin, cola, guarana, neroli, pineapple, apricot, banana, melon, apricot, ume, cherry, raspberry, blackberry, tropical fruit, mango, mangosteen, pomegranate, papaya and so forth. Additional exemplary flavors imparted by a flavoring agent may include a milk flavor, a butter flavor, a cheese flavor, a cream flavor, and a yogurt flavor; a vanilla flavor; tea or coffee flavors, such as a green tea flavor, an oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamon flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; a nut flavor such as an almond flavor, a hazelnut flavor, a macadamia nut flavor, a peanut flavor, a pecan flavor, a pistachio flavor, and a walnut flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor.

In some embodiments, other flavoring agents may include aldehydes and esters such as cinnamyl acetate, cinnamaldehyde, citral diethylacetal, dihydrocarvyl acetate, eugenyl formate, p-methylamisol, and so forth. Examples of aldehyde flavorings may include acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin), and the like.

The flavoring agents may be used in liquid or solid/dried form and may be used individually or in a mixture. When employed in dried form, suitable drying means such as spray drying an oil may be used. Alternatively, the flavoring agent may be absorbed onto water-soluble materials, such as cellulose, starch, sugar, maltodextrin, gum arabic and so forth or may be encapsulated. In still other embodiments, the flavoring agent may be adsorbed onto silicas, zeolites, and the like. The techniques for preparing such dried forms are well-known.

In some embodiments, the flavoring agents may be used in many distinct physical forms. Without being limited thereto, such physical forms may include free forms, such as spray dried, powdered, beaded forms, encapsulated forms, emulsions such as caramel or gum arabic emulsions, and a combination comprising at least one of the foregoing physical forms. The particular amount of the flavoring agent effective for imparting flavor characteristics to the composition may depend upon several factors including the flavor, the flavor impression, and the like.

In another embodiment, a consumable composition may also contain flavor potentiators. Examples of suitable potentiators may include neohesperidin dihydrochalcone, chlorogenic acid, alapyridaine, cynarin, miraculin, glupyridaine, pyridinium-betain compounds, glutamates, such as monosodium glutamate and monopotassium glutamate, neotame, thaumatin, tagatose, trehalose, salts, such as sodium chloride, monoammonium glycyrrhizinate, vanilla extract (in ethyl alcohol), sugar acids, potassium chloride, sodium acid sulfate, hydrolyzed vegetable proteins, hydrolyzed animal proteins, yeast extracts, adenosine monophosphate (AMP), glutathione, nucleotides, such as inosine monophosphate, disodium inosinate, xanthosine monophosphate, guanylate monophosphate, alapyridaine (N-(1-carboxyethyl)-6-(hydroxymethyl)pyridinium-3-ol inner salt), sugar beet extract (alcoholic extract), sugarcane leaf essence (alcoholic extract), curculin, strogin, mabinlin, gymnemic acid, hydroxybenzoic acids, 3-hydrobenzoic acid, 2,4-dihydrobenzoic acid, citrus aurantium, vanilla oleoresin, sugarcane leaf essence, maltol, ethyl maltol, vanillin, licorice glycyrrhizinates, compounds that respond to G-protein coupled receptors (T2Rs and T1Rs) and taste potentiator compositions that impart kokumi, as disclosed in U.S. Pat. No. 5,679,397 to Kuroda et al, which is incorporated in its entirety herein by reference, and a combination comprising any of the foregoing potentiators. “Kokumi” refers to materials that impart “mouthfulness” and “good body.”

Sweetener potentiators, which are a type of flavor potentiator, may enhance the taste of sweetness. In some embodiments, exemplary sweetener potentiators may include monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, hydroxybenzoic acids, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs), and a combination comprising any of the foregoing potentiators.

Some embodiments also may include a sweetening agent to provide a sweet taste to the composition. Sweetening agents may include sugar sweeteners, sugarless sweeteners, and a combination comprising any of the foregoing. Sugar sweeteners generally include saccharides. Suitable sugar sweeteners may include mono-saccharides, di-saccharides and poly-saccharides such as sucrose (sugar), dextrose, maltose, dextrin, xylose, ribose, glucose, mannose, galactose, fructose (levulose), lactose, invert sugar, fructo oligo saccharide syrups, partially hydrolyzed starch, corn syrup solids, such as high fructose corn syrup, and a combination comprising any of the foregoing.

Exemplary sugarless sweetening agents may include sugar alcohols (or polyols), such as glycerol, sorbitol, xylitol, mannitol, galactitol, maltitol, hydrogenated isomaltulose (isomalt), lactitol, erythritol, hydrogenated starch hydrolysate, polyglycitol (e.g., syrup or powder), stevia and a combination comprising any of the foregoing. Exemplary hydrogenated starch hydrolysates may include those disclosed in U.S. Pat. Nos. 3,356,811, 4,279,931 and various hydrogenated glucose syrups and/or powders which contain sorbitol, hydrogenated disaccharides, hydrogenated higher polysaccharides, and a combination comprising any of the foregoing. Hydrogenated starch hydrolysates may be prepared by the controlled catalytic hydrogenation of corn syrups. The resulting hydrogenated starch hydrolysates are mixtures of monomeric, dimeric, and polymeric saccharides. The ratios of these different saccharides may give different hydrogenated starch hydrolysates different properties. Mixtures of hydrogenated starch hydrolysates, such as LYCASIN™, a line of commercially available products manufactured by Roquette Freres of France, and HYSTAR™, a line of commercially available products manufactured by Lonza, Inc., of Fairlawn, N.J., also may be useful.

Some embodiments may include high-intensity sweeteners in the composition. Exemplary high-intensity sweeteners may include: (1) water-soluble sweetening agents such as, for example, dihydrochalcones, monellin, steviosides, rebaudiosides such as rebaudioside A, glycyrrhizin, dihydroflavenol, and sugar alcohols such as sorbitol, mannitol, maltitol, and L-aminodicarboxylic acid aminoalkenoic acid ester amides, such as those disclosed in U.S. Pat. No. 4,619,834, which disclosure is incorporated herein by reference, and a combination comprising any of the foregoing; (2) water-soluble artificial sweeteners such as, for example, soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and a combination comprising any of the foregoing; (3) dipeptide based sweeteners, such as, for example, L-aspartic acid derived sweeteners, such as L-aspartyl-L-phenylalanine methyl ester (Aspartame) and materials described in U.S. Pat. No. 3,492,131, L-alphaaspartyl-N-(2,2,4,4-tetramethyl-3-thietanyl)-D-alaninamide hydrate (Alitame), N-[N-(3,3-dimethylbutyl)-L-aspartyl]-L-phenylalanine 1-methyl ester (Neotame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(I-cyclohexen)-alanine, and a combination comprising any of the foregoing; (4) water-soluble sweeteners derived from naturally occurring water-soluble sweeteners, such as, for example, chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′rideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro4,6,1′1,6′-tetradeoxygalacto-sucrose; and 4,6,1′,6′-tetradeoxy-sucrose, and a combination comprising any of the foregoing; (5) protein based sweeteners such as, for example, thaumaoccous danielli (Thaumatin I and II); and (6) the naturally occurring sweetener monatin (2-hydroxy-2-(indol-3-ylmethyl)-4-aminoglutaric acid) and its derivatives.

Many sweetening agents, including some previously discussed, may be categorized as natural sweeteners such as, for example, L-alanine, arabinose, banana extract, carob, cellobiose, corn syrup (including high fructose corn syrup and corn syrup solids), dextrin, dextrose, Dioscoreophyllum cumminsii (Serendipity Berry), erythritol, fructooligosaccharide (FOS), fructose, (including “liquid fructose”), galactose, glucose, glycine, glycyrrhizin, honey, inulin, isomalt, invert sugar, lactitol, lactose, lo han (lo han kuo; lo han guo; Johan guo; lohan kuo), maltitol, maltodextrin, maltose, mannitol, mannose, maple syrup, molasses, partially hydrogenated starch hydrolysate, partially hydrolyzed starch, polydextrose solution, polyglycitol, raftilose, miraculin (Richardella dulcifica (Miracle Berry)), ribose, rice syrup, sorbitol, sorbose, stevia, stevioside, rebaudioside (including rebaudioside A), sucralose, sucrose, sugar beets, (dehydrated filaments of), D-tagatose, thaumatin, xylitol, xylose, and a combination comprising any of the foregoing.

Sweetening agents may be used individually or as mixtures and may be used in many distinct physical forms well-known in the art to provide an initial burst of sweetness and/or a prolonged sensation of sweetness. Without being limited thereto, such physical forms may include free forms, such as spray dried, powdered, beaded forms, encapsulated forms, and a combination comprising any of the foregoing. In general, an effective amount of sweetener may be utilized to provide a level of sweetness desired, and this amount may vary with the sweetener selected. Suitable amounts for each type of sweetener may be selected by one of ordinary skill in the art without undue experimentation.

In some embodiments a consumable composition may include antioxidant additives such as citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, flavonoids, and a combination comprising any of the foregoing. Other embodiments of a consumable composition may include added amino acids such as L-tryptophan, L-lysine, L-leucine, L-methionine, 2-aminoethanesulfonic acid (taurine), and L-carnitine; creatine; glucuronolactone; inositol; and a combination comprising any of the foregoing.

In some embodiments, a consumable composition may include additives such as caffeine, coloring agents (“colorants”, “colorings”), emulsifiers, food-grade acids, minerals, micronutrients, plant extracts, preservatives, salts including buffering salts, stabilizers, thickening agents, medicaments, and a combination comprising any of the foregoing. Those of ordinary skill in the art will understand that certain additives may meet the definition or function according to more than one of the above-listed additive categories.

Exemplary salts may include alkali or alkaline earth metal chlorides, glutamates, and the like. For example, monosodium glutamate, potassium chloride, sodium chloride, and a combination comprising any of the foregoing salts may be used. The salts may be added to the consumable composition as a flavor potentiator as described above. Food-grade acids for use in certain embodiments of the consumable composition may include, for example, acetic acid, adipic acid, ascorbic acid, butyric acid, citric acid, formic acid, fumaric acid, glyconic acid, lactic acid, malic acid, phosphoric acid, oxalic acid, succinic acid, tartaric acid, and a combination comprising any of the foregoing food-grade acids. The food-grade acid may be added as acidulent to control the pH of the consumable composition and also to provide some preservative properties; or to stabilize the consumable composition. The pH of the consumable composition may also be modified by the addition of food-grade compounds such as ammonium hydroxide, sodium carbonate, potassium carbonate, sodium bicarbonate, and the like, and a combination comprising any of the foregoing. Additionally, the pH of the consumable composition may be adjusted by the addition of carbon dioxide.

In some embodiments, the tartness of the consumable composition may be varied by selecting and combining acids to provide a desired tartness perception. Some factors to consider in determining a desired tartness include, for example, the acid's dissociation constant, solubility, pH, etc. These variables may be measured by measuring the titratable acidity of the consumable composition.

In one embodiment of a consumable composition, a coloring agent may be used in amounts effective to produce a desired color for the composition. Exemplary coloring agents may include pigments, natural food colors and dyes suitable for food, drug and cosmetic applications. A full recitation of all colorants approved by the United States Food and Drug Administration, together with corresponding chemical structures, may be found in the Kirk-Othmer Encyclopedia of Chemical Technology, 3rd Edition, in volume 5 at pages 857-884, which text is incorporated herein by reference.

As classified by the United States Food, Drug, and Cosmetic Act (21 C.F.R. 73), colors may include those exempt from certification colors (sometimes referred to as natural even though they can be synthetically manufactured) and certified colors (sometimes referred to as artificial), and a combination comprising any of the foregoing. In some embodiments, exemplary colors exempt from certification or natural colors may include, for example, annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthinlcapsorbin (E160c), lycopene (E160d), and a combination comprising any of the foregoing.

In some embodiments, exemplary certified colors may include FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminum (E173), silver (E174), gold (E175), pigment rubinellithol rubine BK (E180), calcium carbonate (E170), carbon black (E153), black PN/brilliant black BN (E151), green S/acid brilliant green BS (E142), and a combination comprising any of the foregoing. In some embodiments, certified colors may include FD&C aluminum lakes, which consist of the aluminum salts of FD&C dyes extended on an insoluble substrate of alumina hydrate. Additionally, in some embodiments, certified colors may be included as calcium salts.

In another embodiment, emulsifiers may be added to the consumable composition to prevent separation of the composition components by keeping ingredients dispersed. Emulsifiers may include molecules which have both a hydrophilic part and a hydrophobic part. Emulsifiers may operate at the interface between hydrophilic and hydrophobic materials of the consumable composition to prevent separation of the components of the composition. Suitable emulsifiers for use in the described compositions may include, for example, lecithin (e.g., soy lecithin); mono and di-glycerides of long chain fatty acids, specifically saturated fatty acids, and more specifically, stearic and palmitic acid mono- and diglycerides; mono and di-glycerides of acetic acid, citric acid, tartaric acid, or lactic acid; egg yolks; polysorbates (e.g., polysorbate 20, polysorbate 40, polysorbate 60, polysorbate 65, and polysorbate 80), propylene glycol esters (e.g, propylene glycol monostearate); propylene glycol esters of fatty acids; sorbitan esters (e.g., sorbitan monostearates, sorbitan tristearates, sorbitan monolaurate, sorbitan monooleate), Acacia (gum arabic), sucrose monoesters; polyglycerol esters; polyethoxylated glycerols; and the like, and a combination comprising any of the foregoing emulsifiers.

In yet another embodiment, a consumable composition may include certain components (sometimes referred to as hydrocolloids) that act as thickening agents which may impart added “mouth-feel” to the composition. Exemplary thickening agents may include natural and synthetic gums, for example locust bean gum, guar gum, gellan gum, xanthan gum, gum ghatti, modified gum ghatti, tragacanth gum, carrageenan, and the like; natural and modified starches, for example pregelatinized starch (corn, wheat, tapioca), pregelatinized high amylose-content starch, pregelatinized hydrolyzed starches (maltodextrins, corn syrup solids), chemically modified starches such as pregelatinized substituted starches (e.g., octenyl succinate), and the like; cellulose derivatives, for example carboxymethylcellulose, sodium carboxymethylcellulose, and the like; polydextrose; whey or whey protein concentrate; pectin; gelatin; and a combination comprising any of the foregoing thickening agents.

In some embodiments, a consumable composition may include additional preservatives to provide freshness and to prevent the unwanted growth of bacteria, molds, fungi, or yeast. The addition of a preservative, including antioxidants, may also be used to maintain the composition's color, flavor, or texture. Exemplary preservatives may include benzoic acid alkali metal salts (e.g., sodium benzoate), sorbic acid alkali metal salts (e.g., potassium sorbate), ascorbic acid (Vitamin C), citric acid, calcium propionate, sodium erythorbate, sodium nitrite, calcium sorbate, butylated hydroxyanisole (BHA), butylated hydroxytoluene (BHT), ethylenediaminetetraacetic acid (EDTA), tocopherols (Vitamin E), straight chain polyphosphates, and a combination comprising any of the foregoing preservatives.

In another embodiment, the consumable composition may undergo high pressure homogenization to provide a homogenous composition. Any conventional homogenization equipment can be employed, such as equipment available from APV Gaulin, Alfa-Laval or Niro Soavi. In some embodiments, the consumable composition may be packaged, ready-to-drink, and shelf-stable. Any type of packaging may be used to package the consumable composition including glass bottles, plastic bottles and containers (e.g., polyethylene terephthalate or foil lined ethylene vinyl alcohol), metal cans (e.g., coated aluminum or steel), lined cardboard containers, and the like. Other packaging material known to one of ordinary skill in the art may also be used.

All patents, patent applications, and other references identified by number herein are incorporated herein by reference in their entirety.

Although the foregoing specific details describe certain embodiments of this invention, persons reasonably skilled in the art will recognize that various changes may be made in the details of this invention without departing from the spirit and scope of the invention as defined in the appended claims and considering the doctrine of equivalents. Therefore, it should be understood that this invention is not to be limited to the specific details shown and described herein.

What is claimed is:

1. A shelf-stable consumable composition comprising a Probiotic-Mimicking Element (PME);
said PME being derived from a probiotic;
wherein said PME has a median particle size less than a median particle size of said probiotic.

2. The composition of claim 1 wherein said median particle size of said PME is less than about 0.2 microns.

3. The composition of claim 2 wherein said composition comprises a substantially transparent beverage.

4. The composition of claim 1 wherein said PME comprises at least one of peptides, fatty acids, bacteriocins, cell surface proteins, phospholipids, teichoic acids, and nucleic acids.

5. The composition of claim 1 further comprising a prebiotic.

6. The composition of claim 5 wherein said prebiotic comprises at least one of oligosaccharides, inulin, short-chain fructo-oligosaccharides, xylooligosaccharides, galactooligosaccharides, soyoligosaccharides, and lactulose/lactitol.

7. A method of preparing a shelf-stable consumable composition comprising:
preparing at least one PME having a median particle size that is less than a median particle size of a probiotic from which said at least one PME is derived; and
adding said at least one PME to a consumable composition.

8. The method of claim 7 wherein said median particle size of said at least one PME is less than about 0.2 microns.

9. The method of claim 7 further comprising:
growing bacteria to form said probiotic; and
inactivating said probiotic to form an inactivated probiotic comprising said at least one PME.

10. The method of claim 9 wherein said inactivating comprises at least one of application of heat, pressure, electromagnetic energy, physical maceration, sonic energy, chemicals, and a combination thereof.

11. The method of claim 7 further comprising adding a prebiotic to said composition.

12. A method for promoting digestive health in a subject, comprising:
orally administering to said subject an effective amount of a shelf-stable consumable composition comprising at least one PME;
wherein said at least one PME has a median particle size that is less than a median particle size of a probiotic from which said at least one PME is derived.

13. The method of claim 12 wherein said median particle size of said at least one PME is less than about 0.2 microns.

14. The method of claim 12 wherein said at least one PME comprises at least one of peptides, fatty acids, bacteriocins, cell surface proteins, phospholipids, teichoic acids, and nucleic acids.

15. The method of claim 12 wherein said shelf-stable consumable composition further comprises at least one prebiotic.

16. The method of claim 15 wherein said at least one prebiotic comprises at least one of oligosaccharides, inulin, short-chain fructo-oligosaccharides, xylooligosaccharides, galactooligosaccharides, soyoligosaccharides, and lactulose/lactitol.
